(19) United States
(12) Patent Application Publication
(10) Pub. No.: US 2024/0287426 A1
(43) Pub. Date: Aug. 29, 2024

(54) ELECTROCHEMICAL SYSTEM FOR SIMULTANEOUS CARBON DIOXIDE CAPTURE/RELEASE AND HYDROGEN PRODUCTION

(71) Applicant: UCHICAGO ARGONNE, LLC, Chicago, IL (US)

(72) Inventors: YuPo J. LIN, Naperville, IL (US); Thomas LIPPERT, Woodridge, IL (US)

(73) Assignee: UCHICAGO ARGONNE, LLC, Chicago, IL (US)

(21) Appl. No.: 18/586,922

(22) Filed: Feb. 26, 2024

Related U.S. Application Data
(60) Provisional application No. 63/448,417, filed on Feb. 27, 2023.

(51) Int. Cl. C12M 1/107; C02F 3/00; C12P 5/02; C25B 1/04; C25B 3/03; C25B 3/26; C25B 9/19; C25B 9/73; C25B 11/031; C25B 11/046; C25B 15/08

(52) U.S. Cl. CPC C12M 21/04; C02F 3/005; C12P 5/023; C25B 1/04; C25B 3/03; C25B 3/26; C25B 9/19; C25B 9/73; C25B 11/031; C25B 11/046; C25B 15/087

(57) ABSTRACT

A system and apparatus for biomethanation and removing carbon dioxide from the methane comprises (a) a primary anaerobic digester adapted and arranged to generate a biogas mixture comprising methane and carbon dioxide from organic materials; (b) an electrochemical reactor adapted and arranged to capture carbon dioxide from the biogas as bicarbonate and to generate hydrogen by electrolytic water slitting, and (c) a biomethanation reactor adapted and arranged to convert the bicarbonate and hydrogen from the electrochemical reactor to methane. The electrochemical reactor also acidifies a saline process stream from the biomethanation reactor and returns the acidified process stream back into the biomethanation reactor for pH control in the biomethanation process.

### CROSS-REFERENCE TO RELATED APPLICATIONS

This application claims the benefit of U.S. Provisional Application No. 63/448,417, filed on Feb. 27, 2023, which is incorporated herein by reference in its entirety.

### CONTRACTUAL ORIGIN OF THE INVENTION

The United States Government has rights in this invention pursuant to Contract No. DE-AC02-06CH11357 between the United States Government and UChicago Argonne, LLC representing Argonne National Laboratory.

### FIELD OF THE INVENTION

This invention relates to an electrochemical system for simultaneous carbon dioxide delivery and hydrogen production with in situ pH control, which can be used in biomethanation and other processes.

### BACKGROUND OF THE INVENTION

Efficient capture and removal of carbon dioxide from biologically generated biogas is an essential technology for the development of more environmentally acceptable waste-based energy production systems. Converting carbon dioxide produced in wastewater treatment to energy is a strategy for ensuring water and energy resilience. Using renewable resources such as solar and wind for carbon dioxide utilization is also an important strategy to reduce the impact of greenhouse gasses on climate change. Biological conversion of organic materials such as food waste, wastewater and waste-treatment sludge can be used on a large scale. For example, carbon dioxide produced in anaerobic digesters and flue gasses can be used as feedstocks for methane production in hydrogenotrophic methanogenesis bioreactors (biomethanation).

Although microbial populations in biomethanation reactors are robust with respect to impurities (e.g., H₂S) in the feed streams there are still challenges in producing high-purity methane through biomethanation, including (1) the low aqueous solubility of carbon dioxide and (2) obtaining a suitable, convenient and cost effective source of hydrogen. The low solubility of carbon dioxide leads to low carbon dioxide utilization in bioreactors operated at atmospheric pressure. The source, production cost and storage of hydrogen frequently makes the economics and investment potential of biomethanation unfavorable.

Because of the aforementioned challenges there is an ongoing need for alternative apparatus for carbon dioxide capture and release, and/or biomethanation processes for producing high-purity methane from organic/biological waste streams.

### SUMMARY OF THE INVENTION

An electrochemical reactor for capturing carbon dioxide and producing bicarbonate and hydrogen is described herein. The electrochemical reactor is useful for, among other things, converting biogas to a bicarbonate and hydrogen feed stock for biomethanation, and supplying aqueous bicarbonate to algae ponds, along with an acidic stream for pH regulation. The reactor comprises at least one reactor unit including an electrochemical carbon dioxide capture cell and at least one alkaline water electrolysis (AWE) cell adjacent to the ECC cell; and is referred to herein as an “ECC/AWE” reactor.

The ECC cell comprises a first anode (typically in the form of a porous or non-porous metal plate) flanked by a first electrical insulator on one side thereof and a first bipolar membrane on an opposite side thereof. A cation exchange membrane (CEM) is spaced from the first bipolar membrane to define an acid chamber therebetween. The ECC cell also includes a porous carbon dioxide capture chamber flanked by the CEM on one side thereof and a second bipolar membrane on an opposite side thereof. A cathode (typically in the form of a porous or non-porous metal plate) is positioned adjacent the second bipolar membrane and adjacent to a second electrical insulator. The carbon dioxide capture chamber can comprise a porous ion-exchange resin wafer or a simple porous medium such as polymeric foam, without any ion-exchange character.

The AWE cell comprises an anode flanked by the second electrical insulator of the ECC cell on one side thereof and a porous diaphragm on an opposite side thereof, and a cathode flanked by the diaphragm on one side thereof and another electrical insulator on an opposite side thereof. In some embodiments, the reactor unit comprises multiple AWE units in a stack, which are insulated from each other by the electrical insulator of an adjacent AWE cell.

In addition, the electrochemical reactor includes: a first liquid inlet line in fluid communication with the acid generation chamber between the first bipolar membrane and the CEM of the ECC cell; a first liquid outlet line in fluid communication with the acid generation chamber positioned opposite the first inlet line; a second liquid inlet line in fluid communication with the carbon dioxide capture chamber; a second liquid outlet line in fluid communication with the carbon dioxide capture chamber positioned opposite the second inlet line; and a recirculating loop configured and adapted to recirculate a liquid (e.g., an aqueous alkaline liquid) through or over the anodes and the cathodes during use. A gas inlet line is in fluid communication with the carbon dioxide capture chamber (e.g., for conveying a biogas into the porous medium of the carbon dioxide capture chamber from an anaerobic digester), and a gas vent line is configured and adapted to vent a gas (e.g., carbon dioxide-depleted biogas) from the second liquid outlet line.

A first gas outlet line is in fluid communication with the anode of the ECC cell (e.g., to vent electrochemically generated oxygen formed at the anode); and a second gas outlet line is in fluid communication with the anode of the AWE (e.g., to vent electrochemically generated oxygen formed at the anode). A first gas collection and transport line is in fluid communication with the first and second gas outlet lines (e.g., to collect the oxygen vented from the anodes).

A third gas outlet line is in fluid communication with the cathode of the ECC cell (e.g., to vent electrochemically generated hydrogen formed at the cathode); and a second gas outlet line is in fluid communication with the cathode of the AWE (e.g., to vent electrochemically generated hydrogen formed at the cathode). A second gas collection and transport line is in fluid communication with the third and fourth gas outlet lines (e.g., to collect the hydrogen vented from the cathodes and convey it into a biomethanation reactor as a feedstock for methane production).

The ECC/AWE reactor is useful for any downstream CO₂ utilization system, e.g., in conjunction with an electrochemical catalytic reactor, a bioreactor for methane production, or for supplying bicarbonate and acidic feedstocks to algae ponds. The ECC/AWE reactor is particularly useful for facilitating biomethanation.

When used in a biomethanation system, an alkaline electrolyte solution (e.g., aqueous KOH) is circulated through (in the case of porous electrodes) or over (in the case of non-porous electrodes) the anodes and cathodes, an aqueous saline solution is conveyed from a biomethanation reactor (e.g., a hydrogenotrophic methanogenesis bioreactor) into the acid generation chamber and carbon dioxide capture chamber of the ECC cell; and a biogas comprising methane and carbon dioxide is conveyed into the carbon dioxide capture chamber; while simultaneously applying a voltage across the anodes and cathodes. Hydrogen gas is electrochemically generated from the alkaline solution at the cathodes and oxygen gas is electrochemically generated from the alkaline solution at the anodes, which together drives exchange of sodium cations from the saline solution in the acid generation chamber through the CEM and into the carbon dioxide capture chamber, and exchange of protons from the anode into the acid generation chamber from the first bipolar membrane thereby creating an acidic process stream. This acidic process stream is conveyed out of the acid generation chamber and into the biomethanation reactor via the first liquid outlet line. Carbon dioxide is captured from the biogas entering the carbon dioxide capture chamber and is converted to bicarbonate by hydroxide ion which migrates into the carbon dioxide capture chamber from the second bipolar membrane of the ECC cell. The resulting bicarbonate solution is conveyed out of the carbon dioxide capture chamber through the second liquid outlet line and into the biomethanation reactor. Preferably, the alkaline electrolyte comprises an alkali metal hydroxide such as potassium hydroxide or sodium hydroxide at a concentration of about 20 percent by weight (wt %) to about 40 wt %, more preferably about 25 wt % to about 30 wt %. For biomethanation applications and many algae pond applications, the aqueous saline solution typically comprises sodium chloride at a concentration of about 0.5 g/L to about 5 g/L, preferably about 1 g/L to about 2 g/L. For saline adapted biomethanation applications, a typical sodium chloride concentration in the aqueous saline solution is up to about 35 g/L sodium chloride.

The bicarbonate solution and hydrogen gas that are fed into the biomethanation reactor from the electrochemical reactor act as the carbon dioxide and hydrogen feedstocks for methane generation, and acidic process stream provides pH control for preventing an overly alkaline environment in the biomethanation reactor, so that high methanogenic activity can be sustained. The ratio of delivery of the acidic process stream and the bicarbonate solution into the biomethanation reactor is adjustable to optimize the pH in the biomethanation reactor for optimal methane production. The number of AWE cells in each reactor unit is chosen, in conjunction to the size and capacity of the ECC cell, to generate the optimum stoichiometry of hydrogen to carbon dioxide for biomethanation (e.g., 4 moles of hydrogen gas to 1 mole of carbon dioxide for hydrogenotrophic methanogenesis bioreactors). In some embodiments, the electrochemical reactor comprises multiple reactor units working in tandem so as to efficiently handle the amount of biogas feeding the electrochemical reactor from the anaerobic digester. At the same time, carbon dioxide-depleted methane from the biogas that passes through the carbon dioxide capture chamber is vented from the bicarbonate stream and recovered. The recovered methane boosts the overall yield of methane from the system.

The tandem electrochemical and gas-phase bioreactor system and method described herein provide a number of advantages for biomethanation, including the ability to adjust the ratio of the hydrogen and carbon dioxide feed stocks for optimum biomethanation without using a separate hydrogen gas source. The system generates high-purity renewable methane rather than just biogas (a mixture of methane and carbon dioxide). The acidic process stream generated in the ECC cell also provides automatic pH control for the methane production. The advantages of the electrochemical reactor described herein include downstream pH control for the biomethanation process, which preserves microbial activity; the ability to maintain high alkalinity in the carbon dioxide capture chamber, which provides for rapid capture of carbon dioxide from biogas; stoichiometric production of four moles of hydrogen gas for each mole of bicarbonate (e.g., the overall stoichiometry of the biomethanation process); and maximum methane gas quality. In addition, the system provides as much as a two- to three-fold increase in carbon dioxide residence time in the biomethanation reactor, depending on the biogas composition (i.e., the ratio of carbon dioxide to methane in the biogas), and the electrochemical reactor operates with electric energy similar to standard alkaline water electrolysis systems used for hydrogen and oxygen production.

The primary anaerobic digester of the system can comprise one stage or multiple stage reactors. In some embodiments, the digester is a dual stage reactor that includes (i) an acidogenesis reactor adapted and arranged to biologically convert organic waste and wastewater to soluble volatile fatty acids (VFA), methane, carbon dioxide, and hydrogen; and (ii) an acetoclastic methanogenesis reactor adapted and arranged to biologically convert the VFA to carbon dioxide and methane. The gas transfer line of the dual reactor is in fluid communication with both the acidogenesis reactor and the acetoclastic methanogenesis reactor; and the acidogenesis reactor is in fluid communication with the acetoclastic methanogenesis reactor via a liquid feed line.

In use, VFA, carbon dioxide, hydrogen and methane are generated in the acidogenesis reactor, and VFA-containing liquid from the acidogenesis reactor is fed into the acetoclastic methanogenesis reactor via the liquid feed line. Methane and carbon dioxide are generated in the acetoclastic methanogenesis reactor from the VFA, and carbon dioxide, methane, and hydrogen are transferred from the acidogenesis reactor and the acetoclastic methanogenesis reactor into the inlet end of electrochemical reactor through the gas transfer line.

Optionally, the ECC cell includes a porous gas and liquid flow distributor in contact with the carbon dioxide capture chamber, particularly when the porous medium of the carbon dioxide capture chamber is an ion-exchange resin wafer. The flow distributor is adapted and arranged to laterally distribute gas and liquid within pores thereof prior to entering the carbon dioxide capture chamber of the electrochemical reactor, which improves gas distribution in the carbon dioxide capture chamber. Preferably, the flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the flow distributor and into the carbon dioxide capture chamber of the electrochemical reactor. Optionally, flue gas can be introduced into the electrochemical reactor along with the gases from the anaerobic digester to supplement the carbon dioxide from the digester.

Commercial algae ponds often require enrichment with carbon dioxide or a carbon dioxide source to promote efficient algae growth. In addition, the pH of algae bonds often needs to be adjusted during pond operation. When used in conjunction with algae ponds, the ECC/AWE reactor can supply needed carbon dioxide in the form of bicarbonate solution and acid for pH adjustment, when needed.

The following non-limiting embodiments are provided to describe certain aspects and features of the electrochemical reactors and methods described herein.

Embodiment 1 is an electrochemical reactor for capturing carbon dioxide and bicarbonate and generating hydrogen; the reactor comprising at least one reactor unit including an electrochemical reactor for carbon and hydrogen delivery (ECC) cell and first alkaline water electrolysis (AWE) cell adjacent to the ECC cell;
wherein:
the ECC cell comprises:
(a) a first anode flanked by a first electrical insulator on one side thereof and a first bipolar membrane on an opposite side thereof,
(b) a cation exchange membrane spaced from the first bipolar membrane to define an acid generation chamber therebetween;
(c) a porous carbon dioxide capture chamber flanked by the cation exchange membrane on one side thereof and a second bipolar membrane on an opposite side thereof, and
(d) a first cathode flanked by the second bipolar membrane on one side thereof and a second electrical insulator on an opposite side thereof,
the first AWE cell comprises:
(f) a second anode flanked by the second electrical insulator of the ECC cell on one side thereof, and a porous diaphragm on an opposite side thereof, and
(g) a second cathode flanked by the diaphragm on one side thereof and a third electrical insulator on an opposite side thereof.

Embodiment 2 is the electrochemical reactor of embodiment 1, further comprising:
(i) a first liquid inlet line in fluid communication with the acid generation chamber;
(ii) a first liquid outlet line in fluid communication with the acid generation chamber positioned opposite the first inlet line;
(iii) a second liquid inlet line in fluid communication with the carbon dioxide capture chamber;
(iv) a second liquid outlet line in fluid communication with the carbon dioxide capture chamber positioned opposite the second inlet line;
(v) a recirculating loop configured and adapted to recirculate a liquid through or over the first and second anodes and the first and second cathodes during use;
(vi) a gas inlet line in fluid communication with the carbon dioxide capture chamber;
(vii) a gas vent line configured and adapted to vent a gas from the second liquid outlet line;
(viii) a first gas outlet line in fluid communication with the first anode;
(ix) a second gas outlet line in fluid communication with the second anode;
(x) a third gas outlet line in fluid communication with the first cathode; and
(xi) a fourth gas outlet line in fluid communication with the second cathode.

Embodiment 3 is the electrochemical reactor of embodiment 2, further comprising:
(xii) a first gas collection and transport line in fluid communication the first and second gas outlet lines; and
(xiii) a second gas collection and transport line in fluid communication the third and fourth gas outlet lines.

Embodiment 4 is the electrochemical reactor of any one of embodiments 1 to 3, further comprising a porous gas and liquid flow distributor adjacent the gas inlet line and contacting the carbon dioxide capture chamber; and the porous gas and liquid flow distributor is adapted and arranged to distribute gas entering the carbon dioxide capture chamber within liquid flowing through the carbon dioxide capture chamber when in use.

Embodiment 5 is the electrochemical reactor of embodiment 4, wherein the gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the distributor and into the carbon dioxide capture chamber.

Embodiment 6 is the electrochemical reactor of any one of embodiments 1 to 5, wherein the carbon dioxide capture chamber has a thickness in the range of about 1 to about 20 mm.

Embodiment 7 is the electrochemical reactor of any one of embodiments 1 to 6, wherein the electrochemical reactor comprises a stack of 2 to about 50 reactor units, electrically insulated from each other.

Embodiment 8 is the electrochemical reactor of any one of embodiments 1 to 7, wherein each reactor unit comprises 1 to 10 additional AWE cells stacked with the first AWE cell and electrically insulated from each other.

Embodiment 9 is the electrochemical reactor of embodiment 8, further comprising:
(i) a first liquid inlet line in fluid communication with the acid generation chamber;
(ii) a first liquid outlet line in fluid communication with the acid generation chamber positioned opposite the first inlet line;
(iii) a second liquid inlet line in fluid communication with the carbon dioxide capture chamber;
(iv) a second liquid outlet line in fluid communication with the carbon dioxide capture chamber positioned opposite the second inlet line;
(v) a recirculating loop configured and adapted to recirculate a liquid through or over the first anode, the first cathode, the second anode of each AWE cell and the second cathode of each AWE cell during use;
(vi) a gas inlet line in fluid communication with the carbon dioxide capture chamber;
(vii) a gas vent line configured and adapted to vent a gas from the second liquid outlet line;
(viii) a first gas outlet line in fluid communication with the first anode;
(ix) a second gas outlet line in fluid communication with the second anode of each AWE cell;
(x) a third gas outlet line in fluid communication with the first cathode;
(xi) a fourth gas outlet line in fluid communication with the second cathode of each AWE cell;
(xii) a first gas collection and transport line in fluid communication the first and second gas outlet lines; and
(xiii) a second gas collection and transport line in fluid communication the third and fourth gas outlet lines.

Embodiment 10 is the electrochemical reactor of embodiment 8 or embodiment 9, further comprising a porous gas and liquid flow distributor adjacent the gas inlet line and contacting the carbon dioxide capture chamber; and the porous gas and liquid flow distributor is adapted and arranged to distribute gas entering the carbon dioxide capture chamber within liquid flowing through the carbon dioxide capture chamber when in use.

Embodiment 11 is the electrochemical reactor of embodiment 10, wherein the gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the distributor and into the carbon dioxide capture chamber.

Embodiment 12 is the electrochemical reactor of any one of embodiments 1 to 11, wherein the first and second cathodes and the first and second anodes comprise porous conductive plates.

Embodiment 13 is the electrochemical reactor of embodiment 12, wherein the porous conductive plates are porous plates of a material selected from the group consisting of nickel, titanium, stainless steel, and a Magnéli phase titanium oxide.

Embodiment 14 is a method for biologically generating carbon dioxide-depleted biogas comprising the steps of:
(a) generating a biogas comprising methane and carbon dioxide by anaerobic degradation of biological material in an anaerobic digester;
(b) passing the biogas generated in step (a) through the carbon dioxide capture chamber of the electrochemical reactor of claim 1; recirculating an aqueous alkaline solution through or over the first and second anodes and the first and second cathodes thereof, and delivering an aqueous saline solution from a hydrogenotrophic methanogenesis bioreactor into the acid generation chamber and the carbon dioxide capture chamber of the ECC cell of the reactor, while applying a voltage across the first and second anodes and the first and second cathodes thereof to convert carbon dioxide in the biogas to an aqueous bicarbonate solution in the carbon dioxide capture chamber, to generate hydrogen gas by electrochemical water splitting at the first and second cathodes, and to generate and aqueous acid solution in the acid generation chamber;
(c) venting and collecting the methane from the biogas that passes through the carbon dioxide capture chamber;
(d) passing the hydrogen gas, the aqueous bicarbonate solution, and the aqueous acid solution formed in step (b) into the hydrogenotrophic methanogenesis bioreactor;
(e) generating methane in the hydrogenotrophic methanogenesis bioreactor from the hydrogen gas and carbon dioxide generated from the bicarbonate solution; and
(f) venting and collecting the methane that forms in the hydrogenotrophic methanogenesis bioreactor.

Embodiment 15 is the method of embodiment 14, wherein the anaerobic digester comprises:
(i) an acidogenesis reactor adapted and arranged to biologically convert organic waste and wastewater to soluble volatile fatty acids (VFA), methane, carbon dioxide, and hydrogen; and
(ii) an acetoclastic methanogenesis reactor adapted and arranged to biologically convert the VFA to carbon dioxide and methane;
wherein in use, VFA, carbon dioxide, hydrogen and methane are generated in the acidogenesis reactor; VFA-containing liquid from the acidogenesis reactor is fed into the acetoclastic methanogenesis reactor; methane and carbon dioxide are generated in the acetoclastic methanogenesis reactor, and carbon dioxide and methane are transferred from the acidogenesis reactor and the acetoclastic methanogenesis reactor into the carbon dioxide capture chamber of the electrochemical reactor.

Embodiment 16 is the method of embodiment 14 or 15, wherein the electrochemical reactor comprises a plurality of the electrochemical reactor units and each reactor unit is in fluid communication with the anaerobic digester and the hydrogenotrophic methanogenesis bioreactor such that methane and carbon dioxide from the anaerobic digester flows into the carbon dioxide capture chamber of each reactor unit, the aqueous saline is delivered to the acid generation chamber and carbon dioxide capture chamber of each reactor unit; the aqueous alkaline solution is recirculated through or over the anodes and the cathodes of each reactor unit; and the hydrogen gas, the aqueous bicarbonate solution and the aqueous acid solution generated in each reactor unit is transferred to the hydrogenotrophic methanogenesis bioreactor.

Embodiment 17 is the method of embodiment 16, wherein each reactor unit comprises a porous gas and liquid flow distributor in contact with the carbon dioxide capture chamber thereof, and the gas distributor is adapted and arranged to distribute the carbon dioxide and methane gas from the anaerobic digester within the aqueous alkaline solution circulating through the carbon dioxide capture chamber.

Embodiment 18 is the method of embodiment 17, wherein the porous gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the flow distributor and into the carbon dioxide capture chamber.

Embodiment 19 is the method of any one of embodiments 14 to 18, wherein the first and second cathodes and the first and second anodes comprise porous conductive plates.

Embodiment 20 is the method of embodiment 19, wherein the porous conductive plates are porous plates of a material selected from the group consisting of nickel, titanium, stainless steel, and a Magnéli phase titanium oxide.

The systems, electrochemical reactors, and methods described herein comprise certain novel features and a combination of parts hereinafter fully described, which are illustrated in the accompanying drawings, and particularly pointed out in the appended claims. It is to be understood that various changes in the details may be made without departing from the spirit, or sacrificing any of the advantages of the systems, electrochemical reactors, and methods described herein.

### BRIEF DESCRIPTION OF THE DRAWINGS

FIG. 1 provides a schematic illustration of a system for biologically generating methane gas, comprising a primary anaerobic digester that generates a biogas connected to an electrochemical reactor for removing carbon dioxide from the biogas and electrochemically generating hydrogen, bicarbonate, and acid, and a biomethanation reactor (in particular a hydrogenotrophic methanogenesis bioreactor) for converting the hydrogen and bicarbonate into additional methane.

FIG. 2 provides a more detailed illustration of an embodiment of a system for biologically generating methane as shown in FIG. 1, illustrating details of the electrochemical reactor.

FIG. 3 provides a flow diagram of a method for generating methane with reduced carbon dioxide content.

FIG. 4 provides plots of pH versus time for the for carbon dioxide capture chamber (referred to as the “capture chamber”) and the acidic stream generated in the acid generation chamber of the ECC cell (referred to as the “acid chamber”) during operation of a biomethanation system comprising an ECC/AWE reactor as described herein at current densities of 50 mA/cm² and 100 mA/cm².

FIG. 5 provides plots of pH and of inorganic carbon versus ratio of acid to base in the biomethanation reactor during operation of a biomethanation system comprising an ECC/AWE reactor as described herein.

FIG. 6 provides plots of biogas composition versus time for a biomethanation system comprising an ECC/AWE reactor as described herein.

FIG. 7 provides plots of feedstock delivery versus time for a biomethanation system comprising an ECC/AWE reactor as described herein.

FIG. 8 provides plots of energy consumption versus electrolyte concentration for different current densities of 25 to 100 mA/cm² for operation of an AWE portion of the reactor.

### DETAILED DESCRIPTION OF THE PRESENT INVENTION

The organic fraction of municipal solid waste comprises more than 50% of the almost 300 million tons of municipal solid waste generated in the U.S. per year. This fraction is difficult to recycle, and the majority of this waste is landfilled. An additional 55 million dry tons of water resource recovery facility residuals and manure slurries are produced per year. These potential energy sources are largely untapped as waste-to-energy technologies are currently expensive to build and demanding to operate and maintain. The increase in efficiency and reduction in cost resulting from the successful development of the systems and methods described herein can substantially reduce the gasoline gallon equivalent of renewable methane and propel waste-produced renewable energy to become financially favorable.

The systems and methods described herein couple electrochemical conversion of carbon dioxide and hydrogen generation with biological methanogenesis to efficiently form high purity renewable methane at a significantly higher methane production rate than conventional, state of the art biological methanation processes. The systems and methods utilize three processing stages including anaerobic digestion of organic materials (e.g., wastewater, sludge, food waste, etc.) to produce a biogas, an electrochemical reactor to capture and remove carbon dioxide from the biogas to form aqueous bicarbonate, while simultaneously electrochemically generating hydrogen, and finally hydrogenotrophic methanogenesis to convert the bicarbonate and hydrogen into more methane and the acidic solution generated in the electrochemical reactor provides pH control of the biomethanation. Overall, by combining carbon dioxide capture with hydrogen production and pH control, the reactors, systems and methods described herein enhance the carbon dioxide capture rate from biogas, improve residence time for carbon dioxide in the biomethanation reactor, and enhance stoichiometry control for biomethanation feedstocks compared to known electrochemical carbon dioxide capture and delivery systems.

Anaerobic digestion involves the processes of hydrolysis, acidogenesis, acetogenesis, and methanogenesis of organic materials (e.g., food waste, wastewater, waste-treatment sludge, and the like). Hydrolysis involves solubilization of particulates and depolymerization of complex materials such as proteins and carbohydrates. Acidogenesis is a process in which the hydrolyzed materials are converted to volatile fatty acids (VFAs, low molecular weight organic acids). Acetogenesis is a process in which the VFAs are converted to acetic acid, carbon dioxide and hydrogen. Methanogenesis is a process that utilizes the acetic acid and hydrogen from the acetogenesis process to produce methane and carbon dioxide. These four stages typically occur together in an anaerobic digester.

Anaerobic digestion systems can be separated into an acetogenesis stage and a methanogenesis stage. In acetogenesis, acidogenic microorganisms (acidogens) and hydrogen-producing microorganisms generate hydrogen and VFAs. In methanogenesis, acetogenic microorganisms (acetogens) and methane producing microorganisms (methanogens) convert the VFAs into methane (see, e.g., S. Li, X. Yang, in Handbook of Biofuels Production (Second Edition), 2016).

Hydrolysis utilizes bacteria such as bacteria from the genera Bacillus, Cellulomonas and Eubacterium. Acidogensis involves bacteria from genera such as Propionibacterium, Butyrivibrio and Acetivibrio. Acetogenesis utilizes bacteria from genera such as Clostridia and Acetivibrio, in particular clostridium aceticum, acetobacter woodii and clostridium termautotrophicum. Methanogsesis utilizes microorganisms of the order Archaea, which are phylogenetically distinct from both eukaryotes and bacteria, but are often found in association with anaerobic bacteria. Methanogens belong to the phylum Euryarchaeota in five orders that include Methanobacteriales, Methanococcales, Methanomicrobiales, Methanopyrales, and Methanosarcinales. Methanogens do not utilize oxygen for respiration. Instead, methanogens use carbon as the terminal electron acceptor in methanogenesis. The carbon generally comes from low molecular weight organic compounds. The two well described pathways for methanogenesis involve the use of acetic acid or inorganic carbon dioxide as terminal electron acceptors (see Equations (Eq.) 1 and 2):

CH₃COOH→CH₄+CO₂ Eq. 1

CO₂+4H₂→CH₄+2H₂O Eq. 2

Acetogenesis can be illustrated by Equation 3:

2CO₂+4H₂→CH₃COOH+2H₂O Eq. 3

There are two pathways for methanogenesis. Acetoclastic methanogenesis converts acetate to methane and carbon dioxide by Equation 1 described above.

Acetoclastic methanogens include the Methanosarcinales, such as Methanosarcina and Methanosaeta. Methylotrophic methanogenesis utilizes methanol and methylamine as the source of carbon for methane production. Hydrogenotrophic methanogenesis, which occurs in all five of the Euryarchaeota methanogenic orders described above, use hydrogen for the reduction of carbon dioxide according to Equation 2 described above, or reduction of CO or formate by similar processes.

Anaerobic digesters such as acetogenic reactors and methanogenic reactors are described, e.g., in United Nations Food and Agriculture Organization (FAO) Agricultural Services Bulletin-128, “Renewable biological systems for alternative sustainable energy production”, Chapter 4, 1997, available online at the fao.org website; which is incorporated herein by reference in its entirety.

In the electrochemical reactors described herein, carbon dioxide is captured as bicarbonate by reaction with electrochemically produced hydroxide in the carbon dioxide capture chambers. Preferably, in use, the pH of fluid within the carbon dioxide capture chamber of the electrochemical reactor is maintained at a value of at least about 8 (e.g., about 8 to about 9.5) by the electric potential applied across the anode and the cathode. Typically, the electric potential applied across the anode and cathode is at least above 1.23 Volt (e.g., about 1.23 to about 5 Volts) per reactor cell for a desired current. The actual voltage per reactor cell pair will vary with the thickness of the carbon dioxide capture chamber, as will be evident to those of ordinary skill in the art. Thicker carbon dioxide capture chambers will require higher voltages. Preferably, the voltage per cell pair will be greater than 1.23 V if the wafer thickness is greater than 1 mm. Typically, the carbon dioxide capture chambers will have a thickness in the range of about 1 mm to about 20 mm. However, greater thickness may still work. The determination of an appropriate working voltage is within the level of ordinary skill in the art.

In some preferred embodiments, a porous gas distributor is positioned across the inlet end of each carbon dioxide capture chamber. The distributor comprises a porous polymeric foam or sintered material with interconnected pores positioned in close contact with or integral with the inlet end of each carbon dioxide capture chamber, such that liquid and gas entering the distributor are dispersed laterally through the porous material of the distributor before entering the carbon dioxide capture chamber. The lateral dispersion of the gas in the porous distributor provides an even plug-flow distribution of the gas and fluid, which reduces gas channeling during operation of the electrochemical reactor.

The porous gas and liquid distributors (“porous distributors”) can be included as separate pieces that are arranged in close contact across the inlet end of the carbon dioxide capture chamber. Alternatively, the porous distributors can be integral with (e.g., fused to) the carbon dioxide capture chamber. In preferred embodiments, the porous distributors are formed from a porous polymeric foam (e.g., polyethylene foam). Each porous distributor is adapted for introducing CO₂-containing gas bubbles into an aqueous fluid within the carbon dioxide capture chamber to direct an even distribution of micro-sized gas bubbles into the carbon dioxide capture chamber to facilitate conversion of CO₂ from the biogas into bicarbonate ion. The aqueous fluid and gas are laterally dispersed and mixed together as they pass through the interconnected pores of the distributor and into the wafer. The porous distributor disperses the gas into micro-sized bubbles, and creates an even “plug-flow” distribution in the aqueous fluid carrier entering the carbon dioxide capture chamber.

As used herein, the terms “micro-sized gas bubbles”, “microbubbles”, and grammatical variations thereof, refer to gas bubbles having a diameter of less than about 1000 micrometers. The size (i.e., volume) of the pores of the gas distributor influences the size of gas bubbles produced therein. Preferably, the porous distributor has an average pore diameter of about 100 to about 600 micrometers. While it is understood that the pores of the gas distributor may not be spherical in shape, for convenience, the phrase “average pore size of about 100 micrometers” refers to pores having a volume equivalent to the volume of a sphere having a diameter of 100 micrometers. Such gas and liquid flow distributors are described, e.g., in co-owned U.S. Pat. No. 9,339,764 to YuPo J. Lin et al., which is incorporated herein by reference in its entirety.

Porous solid resin wafer ion exchange materials suitable for use in the ECC/AWE reactors described herein can be prepared in any suitable manner known in the art, such as for example the materials and methods described in U.S. Pat. No. 6,495,014 (Datta et al., incorporated herein by reference in its entirety), U.S. Pat. No. 7,452,920 (Lin et al., incorporated herein by reference in its entirety), and U.S. Pat. No. 7,306,934 (Arora et al.). Commercial resin wafers are also available than can be used or adapted for use in the electrochemical reactors described herein.

The anodes and cathodes typically will comprise a porous or non-porous conductive plate, such as porous or non-porous titanium, nickel, stainless steel, and/or Magnéli phase titanium oxides. The electrical insulators typically comprise fluoropolymer elastomers, rubber-like polymers, and/or plastics. The diaphragms of the AWE cells typically comprise a composite separator material made from polysulfone and ZrO₂.

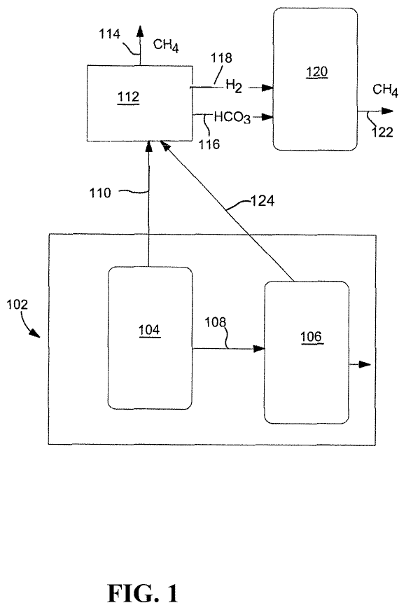

FIG. 1 provides a schematic illustration of a system for biologically generating a methane-containing biogas and removing carbon dioxide from the biogas as bicarbonate, generating hydrogen, and converting the bicarbonate and hydrogen into methane. System 100 comprises a two-stage primary anaerobic digester system 102 including acidogenesis reactor 104 and acetoclastic methanogenesis reactor 106. A liquid feed line 108 provides for transfer of VFA-containing liquid from reactor 104 to reactor 106. Acidogenesis reactor 104 includes a gas transfer line 110 for transferring gases generated in reactor 104 into ECC/AWE electrochemical reactor 112. Acetoclastic methanogenesis reactor 106 also includes a gas transfer line 124 for transferring gases generated in reactor 106 into ECC/AWE electrochemical reactor 112. Reactor 112 includes a gas vent 114 to vent methane for storage and later use, a liquid transfer line 116 and hydrogen transfer line 118, both of which are in fluid communication with hydrogenotrophic methanogenesis bioreactor 120.

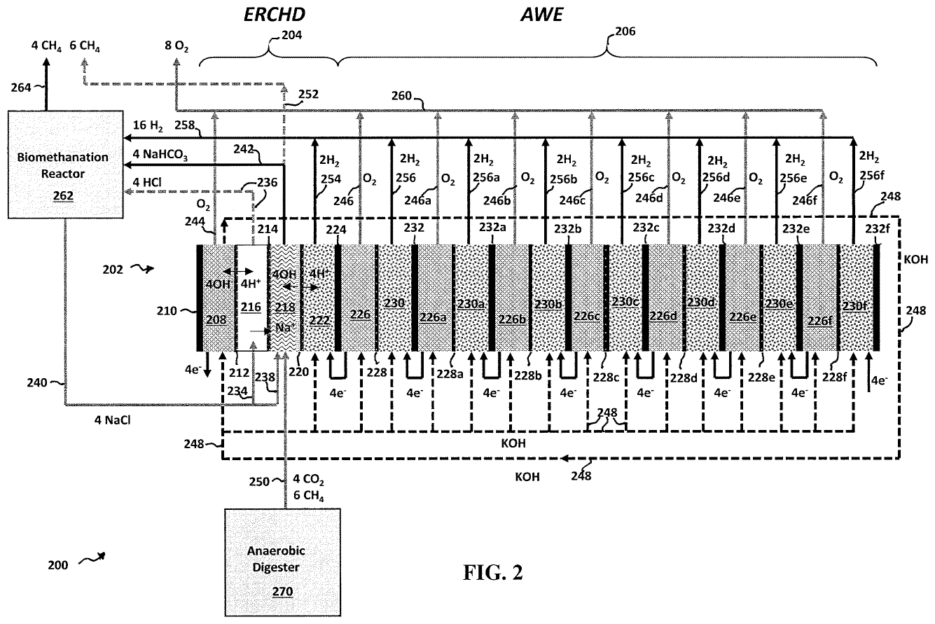

FIG. 2 provides a more detailed illustration of an embodiment of the electrochemical reactor of the system in FIG. 1. In FIG. 2, system 200 comprises electrochemical reactor 202 in fluid communication with anaerobic digester 270 and biomethanation reactor 262. Reactor 202 comprises a reactor unit including an ECC cell 204 and an AWE cell stack 206 comprising a stack of seven individual AWE cells adjacent to the ECC cell 204.

In FIG. 2, the ECC cell comprises an anode 208 flanked by an electrical insulator 210 on one side thereof and a bipolar membrane 212 on an opposite side thereof. A cation exchange membrane 214 is spaced from bipolar membrane 212 to define an acid generation chamber 216 therebetween. A porous carbon dioxide capture chamber 218 is flanked by the cation exchange membrane 214 on one side thereof and a bipolar membrane 220 on an opposite side thereof. A cathode 222 is flanked by bipolar membrane 220 on one side thereof and a second electrical insulator 224 on an opposite side thereof.

The first AWE cell of stack 206 comprises an anode 226 flanked by electrical insulator 224 of the ECC cell on one side thereof, and a porous diaphragm 228 on an opposite side thereof, and a cathode 230 is flanked by diaphragm 228 on one side thereof and an electrical insulator 232 on an opposite side thereof. The six remaining individual AWE cells of stack 206 comprise anodes 226a, b, c, d, e and f, and respective cathodes 230a, b, c, d, e, and f, separated by respective porous diaphragms 228a, b, c, d, e, and f. Each additional AWE cell in stack 206 is insulated by respective electrical insulators 232a, b, c, d, e, and f.

A liquid inlet line 234 is in fluid communication with acid generation chamber 216 for delivering a saline process stream from biomethanation reactor 262 into chamber 216; a liquid outlet line 236 is in fluid communication with acid generation chamber 216 positioned opposite inlet line 234, for delivering an acidic process stream generated in chamber 216 to biomethanation reactor 262; and a liquid inlet line 238 is in fluid communication with carbon dioxide capture chamber 218 for delivering the saline process stream from biomethanation reactor 262 into carbon dioxide capture chamber 218. A liquid outlet line 242 is in fluid communication with carbon dioxide capture chamber 218 positioned opposite inlet line 238, for delivering a bicarbonate process stream generated in carbon dioxide capture chamber 218 into biomethanation reactor 262. A recirculating loop 248 is configured and adapted to recirculate a aqueous KOH electrolyte through or over anodes 208, 226, 226a, 226b, 226c, 226d, 226e, and 226f, and through or over cathodes 222, 230, 230a, 230b, 230c, 230d, 230e, and 230f during use, to support hydrogen production at the cathodes and oxygen production at the anodes.

A gas inlet line 250 is in fluid communication with the carbon dioxide capture chamber 218 to convey biogas comprising carbon dioxide and methane into carbon dioxide capture chamber 218 in order to capture and remove carbon dioxide from the biogas and convert the carbon dioxide to bicarbonate. A gas vent line 252 is configured and adapted to vent carbon dioxide-depleted methane gas from liquid outlet line 242. Gas outlet lines 244, 246, 246a, 246b, 246c, 246d, 246e, and 246f are in fluid communication with anodes 208, 226, 226a, 226b, 226c, 226d, 226e, and 226f and gas collection and transfer line 260 to vent and collect oxygen electrolytically generated at the anodes; and gas outlet lines 254, 256, 256a, 256b, 256c, 256d, 256e, and 256f are fluid communication with the porous cathodes 222, 230, 230a, 230b, 230c, 230d, 230e, and 230f and with a gas collection and transfer line 258 to vent and collect hydrogen electrolytically generated at the cathodes and convey the hydrogen into biomethanation reactor 262. The electrochemical reactor 202 also includes an optional porous gas and liquid flow distributor 272 between gas inlet line 250 and carbon dioxide capture chamber 218; and the porous gas and liquid flow distributor 272 is adapted and arranged to distribute biogas entering carbon dioxide capture chamber 218 within liquid flowing through carbon dioxide capture chamber 218 when in use. In some preferred embodiments gas and liquid distributor 272 is a microporous polymeric foam, which distributes the entering gas and liquid evenly across the carbon dioxide capture chamber 218 to improve the uniformity of flow within the wafer.

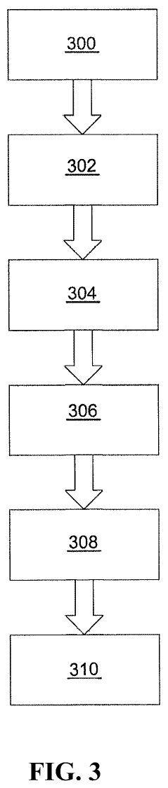

Referring now to FIG. 3, a flow diagram of a method for generating renewable methane from organic materials is provided. Step 300 involves generating a biogas comprising methane and carbon dioxide by anaerobic degradation of biological material in a biomethanation reactor. Step 302 is passing the biogas generated in step 300 and saline solution from the biomethanation reactor through an electrochemical reactor comprising an ECC cell and adjacent AWE cells as described herein, while applying a voltage across the anodes and the cathodes of the electrochemical reactor to convert carbon dioxide in the biogas to bicarbonate and to generate hydrogen gas by electrochemical water splitting at the cathodes, and the combination of ECC and AWE cells is balanced to stoichiometrically deliver back into the biomethanation reactor one mole of bicarbonate and four moles of hydrogen per mole of carbon dioxide generated by the biomethanation reactor, which is the optimal stoichiometry for methane production. Step 304 is venting and collecting the methane from the biogas that passes through the electrochemical reactor. Step 306 is passing the hydrogen, a stream of acidic water, and aqueous alkaline bicarbonate formed in step 302 into a hydrogenotrophic methanogenesis bioreactor. Step 308 is generating methane from the hydrogen and bicarbonate in the hydrogenotrophic methanogenesis bioreactor. And step 310 is venting and collecting the methane that forms in the hydrogenotrophic methanogenesis bioreactor.

The following non-limiting examples are provided to illustrate certain embodiments, features, and/or advantages of the reactors, systems and methods described herein.

### Example 1. Evaluation of Electrochemical Removal of Carbon Dioxide from a Gas Stream with Simultaneous Generation of Hydrogen and pH Control in an Electrochemical Reactor

An ECC/AWE electrochemical reactor consisting of an ECC cell (including one CO₂ capture chamber and one acid generation chamber) and an adjacent AWE cell including a cathodic chamber for H₂ generation and an anodic chamber for O₂ generation was prepared. The ECC/AWE reactor was evaluated using a surrogate biogas feed (35% CO₂ and 65% CH₄), mixed with aqueous NaCl under an applied electric field. The electrolyte used in the AWE was KOH.

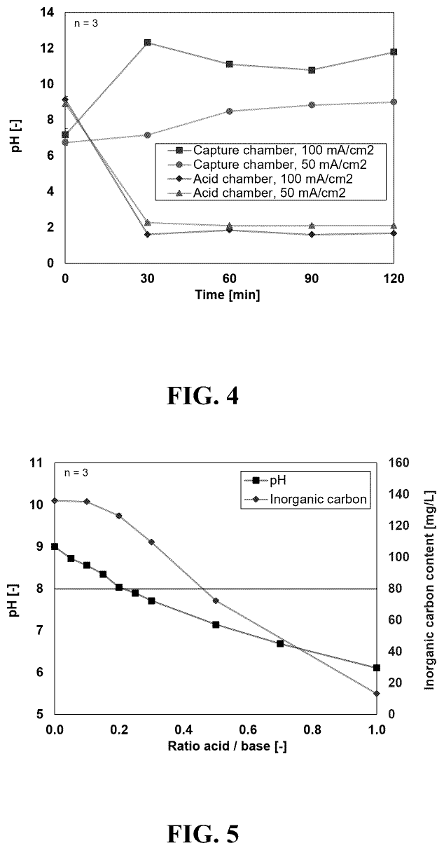

FIG. 4 provides plots of pH versus time for the for carbon dioxide capture chamber (referred to as the “capture chamber”) and the acidic stream generated in the acid generation chamber (referred to as the “acid chamber”) of the ECC cell during operation of the ECC/AWE reactor at current densities of 50 mA/cm² and 100 mA/cm² and NaCl concentrations of 5 g/L. The results were collected by measuring the pH using portable pH meter (Fisher ACUMET AP 62 portable pH meter) of the aqueous samples taken from the basic and acid generation chambers. The results in FIG. 4 show that the ECC can generate both alkaline and acidic streams to fulfill its purpose of carbon dioxide capture and pH control in the downstream biomethanation reactor, respectively.

FIG. 5 provides plots of pH and of inorganic carbon versus ratio of acid to base in the biomethanation reactor during operation of a biomethanation system comprising the ECC/AWE. The applied current density and NaCl concentration were 100 mA/cm² and 5 g/L, respectively. The results were collected by analyzing the inorganic carbon (IC) content in the liquid samples using a TOC analyzer (Shimadzu TOC-V CSN). The results in FIG. 5 show that the pH level in the biomethanation reactor can be successfully and precisely adjusted by merging alkaline and acidic streams.

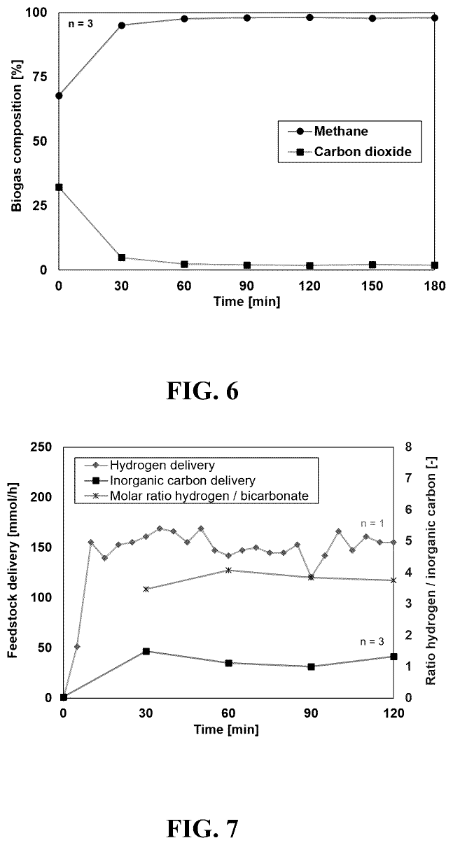

FIG. 6 provides plots of biogas composition versus time for a biomethanation system comprising the ECC/AWE reactor. The employed biogas had a starting composition of 65% methane and 35% carbon dioxide. The results were collected by measuring gas compositions using gas chromatography (GC, Shimadzu GC 2014 TCD-FID), in the gas samples collected from influent and effluent of the basic chamber of ECC and cathodic chamber of the AWE. The results in FIG. 6 show that the ECC/AWE reactor is able to successfully purify biogas to high-purity methane.

FIG. 7 provides plots of feedstock delivery versus time for a biomethanation system comprising the ECC/AWE reactor. The applied current density and NaCl concentration were 100 mA/cm² and 5 g/L, respectively. The results were collected by measuring the gas samples by aforementioned GC analysis and the liquid samples by aforementioned TOC analysis. The results in FIG. 7 show that the ECC/AWE reactor is able to deliver hydrogen and bicarbonate in a molar ratio of 4:1 which represents the ideal molar ratio for biomethanation.

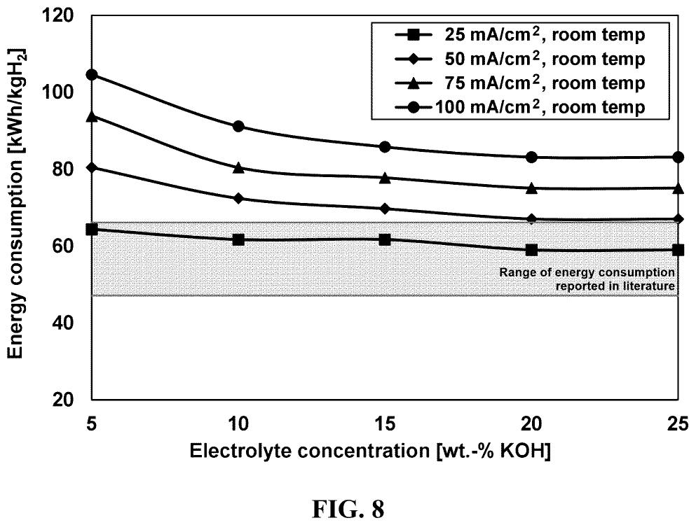

FIG. 8 provides plots of energy consumption of the AWE part excluding the ECC versus electrolyte concentration for different current densities of 25 to 100 mA/cm² at which the ECC/AWE reactor might operate. Employed KOH concentrations ranged from 5 to 25 wt %. The results were collected by H₂ production rate measured by aforementioned GC analysis and the power was calculated from the applied current and voltage of the entire cell during the operation. The results in FIG. 8 show that the energy consumption of the AWE cells described herein were within the range AWE reactors used for alkaline water splitting described in the literature. Instead of using an entire electrochemical system just to capture CO₂, the ECC/AWE reactor is operated similarly to an industrial electrolyzer with small extra energy input to enable capturing the carbon dioxide from biogas and releasing the carbon dioxide from bicarbonate generated by the reactor using the acidic stream, as feedstock for processes utilizing carbon dioxide.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An electrochemical reactor for capturing carbon dioxide and bicarbonate and generating hydrogen; the reactor comprising at least one reactor unit including an electrochemical carbon dioxide capture (ECC) cell and first alkaline water electrolysis (AWE) cell adjacent to the ECC cell;
wherein:
the ECC cell comprises:
(a) a first anode flanked by a first electrical insulator on one side thereof and a first bipolar membrane on an opposite side thereof;
(b) a cation exchange membrane spaced from the first bipolar membrane to define an acid generation chamber therebetween;
(c) a porous carbon dioxide capture chamber flanked by the cation exchange membrane on one side thereof and a second bipolar membrane on an opposite side thereof; and
(d) a first cathode flanked by the second bipolar membrane on one side thereof and a second electrical insulator on an opposite side thereof,
the first AWE cell comprises:
(f) a second anode flanked by the second electrical insulator of the ECC cell on one side thereof, and a porous diaphragm on an opposite side thereof; and
(g) a second cathode flanked by the diaphragm on one side thereof and a third electrical insulator on an opposite side thereof.

2. The electrochemical reactor of claim 1, further comprising:
(i) a first liquid inlet line in fluid communication with the acid generation chamber;
(ii) a first liquid outlet line in fluid communication with the acid generation chamber positioned opposite the first inlet line;
(iii) a second liquid inlet line in fluid communication with the carbon dioxide capture chamber;
(iv) a second liquid outlet line in fluid communication with the carbon dioxide capture chamber positioned opposite the second inlet line;
(v) a recirculating loop configured and adapted to recirculate a liquid through or over the first and second anodes and the first and second cathodes during use;
(vi) a gas inlet line in fluid communication with the carbon dioxide capture chamber;
(vii) a gas vent line configured and adapted to vent a gas from the second liquid outlet line;
(viii) a first gas outlet line in fluid communication with the first anode;
(ix) a second gas outlet line in fluid communication with the second anode;
(x) a third gas outlet line in fluid communication with the first cathode; and
(xi) a fourth gas outlet line in fluid communication with the second cathode.

3. The electrochemical reactor of claim 2, further comprising:
(xii) a first gas collection and transport line in fluid communication the first and second gas outlet lines; and
(xiii) a second gas collection and transport line in fluid communication the third and fourth gas outlet lines.

4. The electrochemical reactor of claim 2, further comprising a porous gas and liquid flow distributor adjacent the gas inlet line and contacting the carbon dioxide capture chamber; and the porous gas and liquid flow distributor is adapted and arranged to distribute gas entering the carbon dioxide capture chamber within liquid flowing through the carbon dioxide capture chamber when in use.

5. The electrochemical reactor of claim 4, wherein the gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the distributor and into the carbon dioxide capture chamber.

6. The electrochemical reactor of claim 1, wherein the carbon dioxide capture chamber has a thickness in the range of about 1 to about 20 mm.

7. The electrochemical reactor of claim 1, wherein the electrochemical reactor comprises a stack of 2 to about 50 reactor units, electrically insulated from each other.

8. The electrochemical reactor of claim 1, wherein each reactor unit comprises 1 to 10 additional AWE cells stacked with the first AWE cell and electrically insulated from each other.

9. The electrochemical reactor of claim 8, further comprising:
(i) a first liquid inlet line in fluid communication with the acid generation chamber;
(ii) a first liquid outlet line in fluid communication with the acid generation chamber positioned opposite the first inlet line;
(iii) a second liquid inlet line in fluid communication with the carbon dioxide capture chamber;
(iv) a second liquid outlet line in fluid communication with the carbon dioxide capture chamber positioned opposite the second inlet line;
(v) a recirculating loop configured and adapted to recirculate a liquid through or over the first anode, the first cathode, the second anode of each AWE cell and the second cathode of each AWE cell during use;
(vi) a gas inlet line in fluid communication with the carbon dioxide capture chamber;
(vii) a gas vent line configured and adapted to vent a gas from the second liquid outlet line;
(viii) a first gas outlet line in fluid communication with the first anode;
(ix) a second gas outlet line in fluid communication with the second anode of each AWE cell;
(x) a third gas outlet line in fluid communication with the first cathode;
(xi) a fourth gas outlet line in fluid communication with the second cathode of each AWE cell;
(xii) a first gas collection and transport line in fluid communication the first and second gas outlet lines; and
(xiii) a second gas collection and transport line in fluid communication the third and fourth gas outlet lines.

10. The electrochemical reactor of claim 8, further comprising a porous gas and liquid flow distributor adjacent the gas inlet line and contacting the carbon dioxide capture chamber; and the porous gas and liquid flow distributor is adapted and arranged to distribute gas entering the carbon dioxide capture chamber within liquid flowing through the carbon dioxide capture chamber when in use.

11. The electrochemical reactor of claim 10, wherein the gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the distributor and into the carbon dioxide capture chamber.

12. The electrochemical reactor of claim 1, wherein the first and second cathodes and the first and second anodes comprise porous conductive plates.

13. The electrochemical reactor of claim 12, wherein the porous conductive plates are porous plates of a material selected from the group consisting of nickel, titanium, stainless steel, and a Magnéli phase titanium oxide.

14. A method for biologically generating carbon dioxide-depleted biogas comprising the steps of:
(a) generating a biogas comprising methane and carbon dioxide by anaerobic degradation of biological material in an anaerobic digester;
(b) passing the biogas generated in step (a) through the carbon dioxide capture chamber of the electrochemical reactor of claim 1; recirculating an aqueous alkaline solution through or over the first and second anodes and the first and second cathodes thereof, and delivering an aqueous saline solution from a hydrogenotrophic methanogenesis bioreactor into the acid generation chamber and the carbon dioxide capture chamber of the ECC cell of the reactor, while applying a voltage across the first and second anodes and the first and second cathodes thereof to convert carbon dioxide in the biogas to an aqueous bicarbonate solution in the carbon dioxide capture chamber, to generate hydrogen gas by electrochemical water splitting at the first and second cathodes, and to generate and aqueous acid solution in the acid generation chamber;
(c) venting and collecting the methane from the biogas that passes through the carbon dioxide capture chamber;
(d) passing the hydrogen gas, the aqueous bicarbonate solution, and the aqueous acid solution formed in step (b) into the hydrogenotrophic methanogenesis bioreactor;
(e) generating methane in the hydrogenotrophic methanogenesis bioreactor from the hydrogen gas and carbon dioxide generated from the bicarbonate solution; and
(f) venting and collecting the methane that forms in the hydrogenotrophic methanogenesis bioreactor.

15. The method of claim 14, wherein the anaerobic digester comprises:
(i) an acidogenesis reactor adapted and arranged to biologically convert organic waste and wastewater to soluble volatile fatty acids (VFA), methane, carbon dioxide, and hydrogen; and
(ii) an acetoclastic methanogenesis reactor adapted and arranged to biologically convert the VFA to carbon dioxide and methane;
wherein in use, VFA, carbon dioxide, hydrogen and methane are generated in the acidogenesis reactor; VFA-containing liquid from the acidogenesis reactor is fed into the acetoclastic methanogenesis reactor; methane and carbon dioxide are generated in the acetoclastic methanogenesis reactor, and carbon dioxide and methane are transferred from the acidogenesis reactor and the acetoclastic methanogenesis reactor into the carbon dioxide capture chamber of the electrochemical reactor.

16. The method of claim 14, wherein the electrochemical reactor comprises a plurality of the electrochemical reactor units and each reactor unit is in fluid communication with the anaerobic digester and the hydrogenotrophic methanogenesis bioreactor such that methane and carbon dioxide from the anaerobic digester flows into the carbon dioxide capture chamber of each reactor unit, the aqueous saline is delivered to the acid generation chamber and carbon dioxide capture chamber of each reactor unit; the aqueous alkaline solution is recirculated through or over the anodes and the cathodes of each reactor unit; and the hydrogen gas, the aqueous bicarbonate solution, and the aqueous acid solution generated in each reactor unit is transferred to the hydrogenotrophic methanogenesis bioreactor.

17. The method of claim 16, wherein each reactor unit comprises a porous gas and liquid flow distributor in contact with the carbon dioxide capture chamber thereof; and the gas distributor is adapted and arranged to distribute the carbon dioxide and methane gas from the anaerobic digester within the aqueous alkaline solution circulating through the carbon dioxide capture chamber.

18. The method of claim 17, wherein the porous gas and liquid flow distributor includes interconnected pores having an average pore size in the range of about 100 to about 600 micrometers, and is adapted and arranged to laterally distribute the liquid and gas bubbles having an average diameter in the range of about 100 to about 600 micrometers throughout the flow distributor and into the carbon dioxide capture chamber.

19. The method of claim 14, wherein the first and second cathodes and the first and second anodes comprise porous conductive plates.

20. The method of claim 19, wherein the porous conductive plates are porous plates of a material selected from the group consisting of nickel, titanium, stainless steel, and a Magnéli phase titanium oxide.
